(19) United States
(12) Patent Application Publication
(10) Pub. No.: US 2024/0287406 A1
(43) Pub. Date: Aug. 29, 2024

(54) FRAGRANCE COMPOSITION

(71) Applicant: Coty Inc., New York, NY (US)

(72) Inventors: Sylvain Solinas, Gex (FR); Beverly Oberti, Prévessin-Moëns (FR)

(21) Appl. No.: 18/568,722

(22) PCT Filed: Jun. 30, 2022
(86) PCT No.: PCT/US22/35773

§ 371 Date: Dec. 8, 2023

Related U.S. Application Data
(60) Provisional application No. 63/217,977, filed on Jul. 2, 2021.

(51) Int. Cl. C11B 9/00; A61K 8/41; A61K 8/49; A61Q 13/00

(52) U.S. Cl. CPC C11B 9/0076; A61K 8/41; A61K 8/4973; A61Q 13/00

(57) ABSTRACT

Various embodiments disclosed relate to a fragrance composition. The fragrance composition includes a fragrance component. The fragrance component further includes a solvent component comprising a compound according to Formula I: wherein R¹, R², and R³ are independently selected from the group consisting of —H, —OH, −CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof.

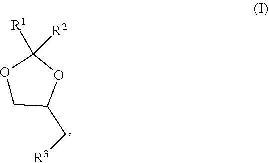

### CROSS-REFERENCE TO RELATED APPLICATION

This application claims the benefit of priority to U.S. Provisional Patent Application Ser. No. 63/217,977 entitled “FRAGRENCE COMPOSITION,” filed Jul. 2 2021, the disclosure of which is incorporated herein in its entirety by reference.

### BACKGROUND

Fragrance compositions with improved fragrance character are desired. However, the solvent environment that a fragrance component is disposed in can lead to undesired consequences. For example, the solvent can cause a side reaction that can lead to the production of bad odors that make a fragrance composition unsuitable.

### SUMMARY OF THE INVENTION

Various embodiments disclosed relate to a fragrance composition. The fragrance composition includes a fragrance component. The fragrance component further includes a solvent component comprising a compound according to Formula I:

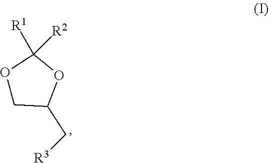

wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof.

Various embodiments disclosed relate to a fragrance composition. The fragrance composition includes a fragrance component. The fragrance composition further includes a solvent component comprising a compound according to Formula I:

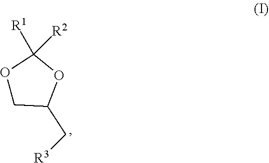

wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof. The fragrance composition further includes a buffer system. The fragrance composition optionally further includes a colorant.

Various embodiments disclosed relate to a fragrance composition. The fragrance composition includes a fragrance component in a range of from about 2 wt % to about 20 wt % of the fragrance composition. The fragrance composition further includes a solvent component that includes a mixture of 2,2-dimethyl-1,3-dioxolan-4-ylmethanol and ethanol and the 2,2-dimethyl-1,3-dioxolan-4-ylmethanol is a major component of the mixture. The fragrance composition further includes a buffer system. The fragrance composition optionally further includes a colorant. The fragrance composition comprises less than about 15 wt % of water and a pH of the fragrance composition is below 7.

Various embodiments disclosed relate to an assembly. The assembly includes a container. A fragrance composition is at least partially disposed in the container. The fragrance component further includes a solvent component comprising a compound according to Formula I.

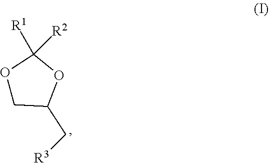

wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “of” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group. A hydrocarbylene group is a diradical hydrocarbon, e.g., a hydrocarbon that is bonded at two locations.

According to various embodiments of the present disclosure a fragrance composition can have a significantly reduced amount of ethanol compared to conventional fragrance compositions.

Various embodiments of the present relate to a fragrance composition. The fragrance composition can include a fragrance component, a solvent component, a buffer system, and a colorant.

The fragrance component can be any suitable component or mixture of components. The fragrance component can be present in a range of from about 2 wt % to about 20 wt % of the fragrance composition, about 5 wt % to about 15 wt % of the fragrance composition, less than, equal to, or greater than about 2 wt %, 3, 4, 5, 6, 7, 8, 9, 10, 11, 12, 13, 14, 15, 16, 17, 18, 19, or about 20 wt % of the fragrance composition.

In some embodiments, the fragrance component can be characterized as having a vapor pressure less than 0.001 Torr at 25° C. In some further embodiments, the fragrance component can be characterized as having a vapor pressure greater than or equal to 0.001 Torr at 25° C. Examples of suitable fragrance components include Cyclopentaneacetic acid, 3-oxo-2-(2Z)-2-penten-1-yl-, methyl ester, (1R,2R)—; 2-Buten-1-ol, 2-methyl-4-(2,2,3-trimethyl-3-cyclopenten-1-yl)-; Ethanone, 1-(2-naphthalenyl)-; 3-Decanone, 1-hydroxy-; Cyclopropanemethanol, 1-methyl-2-[(1,2,2-trimethylbicyclo[3.1.0]hex-3-yl)methyl]-; Benzaldehyde, 3-ethoxy-4-hydroxy-; 2H-1,5-Benzodioxepin-3(4H)-one, 7-methyl-; 2-Butanol, 1-[[2-(1,1-dimethylethyl)cyclohexyl]oxy]-; Spiro[5.5]undec-8-en-1-one, 2,2,7,9-tetramethyl-; Cyclopentaneacetic acid, 3-oxo-2-pentyl-, methyl ester, (1R,2R)-rel-; Cyclopentaneacetic acid, 3-oxo-2-pentyl-, methyl ester; Octanal, 2-(phenylmethylene)-; Cyclopentanecarboxylic acid, 2-hexyl-3-oxo-, methyl ester; 3-Cyclopentene-1-butanol, α,β,2,2,3-pentamethyl-; Cyclopentanone, 2-(3,7-dimethyl-2,6-octadien-1-yl)-; 1,6,10-Dodecatrien-3-ol, 3,7,11-trimethyl-; 2-Pentenenitrile, 3-methyl-5-phenyl-, (2Z)—; Benzenepropanenitrile, 4-ethyl-α,α-dimethyl-; 1H-3a,7-Methanoazulen-6-ol, octahydro-3,6,8,8-tetramethyl-, (3R,3aS,6R,7R,8aS)-; Ethanone, 1-(1,2,3,4,5,6,7,8-octahydro-2,3,8,8-tetramethyl-2-naphthalenyl)-; Propanoic acid, 2-methyl-, 4-formyl-2-methoxyphenyl ester; 1,6-Heptadien-3-one, 1-(2,6,6-trimethyl-2-cyclohexen-1-yl)-; Benzoic acid, 2-hydroxy-, hexyl ester; Benzoic acid, phenyl ester; Cyclohexanepropanol, 2,2,6-trimethyl-α-propyl-, (1R,6S)—; Cyclohexanepropanol, 2,2,6-trimethyl-α-propyl-; Benzoic acid, 2-hydroxy-, 3-methyl-2-buten-1-yl ester; 2H-1,5-Benzodioxepin-3(4H)-one, 7-(1-methylethyl)-; Butanal, 4-(octahydro-4,7-methano-5H-inden-5-ylidene)-; Cyclopenta[g]-2-benzopyran, 1,3,4,6,7,8-hexahydro-4,6,6,7,8,8-hexamethyl-; Cyclopentanone, 2-[2-(4-methyl-3-cyclohexen-1-yl)propyl]-; 2(3H)-Naphthalenone, 4,4a,5,6,7,8-hexahydro-4,4a-dimethyl-6-(1-methylethenyl)-, (4R,4aS,6R)—; 2-Propenoic acid, 3-phenyl-, pentyl ester; 4H-Pyran-4-one, 3-hydroxy-2-methyl-; 1-Propanol, 2-methyl-3-[(1,7,7-trimethylbicyclo[2.2.1]hept-2-yl)oxyl]-; 1-Naphthalenol, 1,2,3,4,4a,5,8,8a-octahydro-2,2,6,8-tetramethyl-; 2-Butenoic acid, 2-methyl-, (2E)-3,7-dimethyl-2,6-octadien-1-yl ester, (2E)-; 1,3-Dioxane, 2-(2,4-dimethyl-3-cyclohexen-1-yl)-5-methyl-5-(1-methylpropyl)-; Nonadecane; 4-Penten-2-ol, 3-methyl-5-(2,2,3-trimethyl-3-cyclopenten-1-yl)-; Propanoic acid, 2-methyl-, 2-methyl-4-oxo-4H-pyran-3-yl ester; 2-Buten-1-ol, 2-ethyl-4-(2,2,3-trimethyl-3-cyclopenten-1-yl)-; 1,6-Methanonaphthalen-1(2H)-ol, octahydro-4,8a,9,9-tetramethyl-, (1R,4S,4aS,6R,8aS)-; 2H-1, 5-Benzodioxepin-3(4H)-one, 7-(1,1-dimethylethyl)-; Benzoic acid, phenylmethyl ester; 8-Cyclohexadecen-1-one; Benzoic acid, 2-hydroxy-, (3Z)-3-hexen-1-yl ester; 4H-Pyran-4-one, 2-ethyl-3-hydroxy-; Cyclopentadecanone, 3-methyl-; Benzoic acid, 2-hydroxy-, phenylmethyl ester; 6,8-Nonadien-3-one, 2,4,4,7-tetramethyl-, oxime; Benzoic acid, 2-hydroxy-, cyclohexyl ester; Benzene, [2-(dimethoxymethyl)-1-hepten-1-yl]-; 3-Cyclopentene-1-butanol, β,2,2,3-tetramethyl-δ-methylene-; 4-Penten-1-one, 1-spiro[4.5]dec-7-en-7-yl-; Acetic acid, 2-(1-oxopropoxy)-, 1-(3,3-dimethylcyclohexyl)ethyl ester; 2-Penten-1-ol, 5-[(1R,3R,6S)-2,3-dimethyltricyclo[2.2.1.02,6]hept-3-yl]-2-methyl-, (2Z)—; 4-Penten-2-ol, 3,3-dimethyl-5-(2,2,3-trimethyl-3-cyclopenten-1-yl)-; 5,8-Methano-2H-1-benzopyran-2-one, 6-ethylideneoctahydro-; 4-Cyclopentadecen-1-one, (4Z)—; Ethanone, 1-[(3R,3aR,7R,8aS)-2,3,4,7,8,8a-hexahydro-3,6,8,8-tetramethyl-1H-3a,7-methanoazulen-5-yl]-; 1,3-Dioxolane, 2,4-dimethyl-2-(5,6,7,8-tetrahydro-5,5,8,8-tetramethyl-2-naphthalenyl)-; Oxacyclohexadecan-2-one; 1-Propanol, 2-[1-(3,3-dimethylcyclohexyl)ethoxy]-2-methyl-, 1-propanoate; 5-Cyclopentadecen-1-one, 3-methyl-; 2-Penten-1-ol, 2-methyl-5-[(1S,2R,4R)-2-methyl-3-methylenebicyclo[2.2.1]hept-2-yl]-, (2Z)—; 2H-1,5-Benzodioxepin-3(4H)-one, 7-(3-methylbutyl)-; Ethanone, 1-(2,6,10-trimethyl-2,5,9-cyclododecatrien-1-yl)-; 1H-3a,6-Methanoazulene-3-methanol, octahydro-7,7-dimethyl-8-methylene-, (3S,3aR,6R,8aS)-; Benzeneacetonitrile, α-cyclohexylidene-; Benzoic acid, 2-[(2-methylpentylidene)amino]-, methyl ester; Benzoic acid, 2-phenylethyl ester; 5-Cyclohexadecen-1-one; Cyclohexanol, 4-(1,7,7-trimethylbicyclo[2.2.1]hept-2-yl)-; 3-Cyclohexene-1-carboxaldehyde, 4-(4-hydroxy-4-methylpentyl)-; Ethanone, 1-(5,6,7,8-tetrahydro-3,5,5,6,8,8-hexamethyl-2-naphthalenyl)-; Ethanone, 1-(5,6,7,8-tetrahydro-3,5,5,6,8,8-hexamethyl-2-; 2-Cyclopentadecen-1-one, 3-methyl-; Oxacycloheptadecan-2-one; Benzeneacetic acid, 4-methylphenyl ester; Benzeneacetic acid, 2-phenylethyl ester; Cyclododecaneethanol, β-methyl-; 2-Propenoic acid, 3-phenyl-, phenylmethyl ester; Benzoic acid, 2,4-dihydroxy-3,6-dimethyl-, methyl ester; Naphtho[2,1-b]furan-6(7H)-one, 8,9-dihydro-1,5,8-trimethyl-, (8R)—; Benzeneacetic acid, (4-methoxyphenyl)methyl ester; Benzene, 2-methoxy-1-(phenylmethoxy)-4-(1-propen-1-yl)-; Benzeneacetic acid, (2E)-3,7-dimethyl-2,6-octadien-1-yl ester; Oxacyclohexadec-12-en-2-one, (12E)-; Benzoic acid, 2-hydroxy-, 2-phenylethyl ester; 2-Propenoic acid, 3-phenyl-, 1-ethenyl-1,5-dimethyl-4-hexen-1-yl ester; Oxacycloheptadec-10-en-2-one; Oxacycloheptadec-8-en-2-one, (8Z)—; 1,7-Dioxacycloheptadecan-8-one; 7-Octen-2-ol, 8-(1H-indol-1-yl)-2,6-dimethyl-; 1,4-Dioxacyclohexadecane-5,16-dione; 1,4-Dioxacycloheptadecane-5,17-dione; Phenol, 4-[3-(benzoyloxy)-1-propen-1-yl]-2-methoxy-; Benzoic acid, 2-[(1-hydroxy-3-phenylbutyl)amino]-, methyl ester; and combinations thereof. Further examples of suitable fragrance components can include Formic acid, methyl ester; Methane, 1,1′-thiobis-; Acetic acid ethyl ester; Propanoic acid, ethyl ester; Acetic acid, 2-methylpropyl ester; Butanoic acid, ethyl ester; 1-Butanol; Butanoic acid, 2-methyl-, ethyl ester; 1-Butanol, 3-methyl-, 1-acetate; Butanoic acid, 2-methyl-, 1-methylethyl ester; 2-Heptanone; 2-Hexenal, (2E)-; 1-Butanol, 3-methyl-; 2-Buten-1-ol, 3-methyl-, 1-acetate; 1,3-Dioxolane-2-methanamine, N-methyl-; Bicyclo[3.1.1]hept-2-ene, 2,6,6-trimethyl-, (1R,5R)—; Bicyclo[2.2.1]heptane, 2,2-dimethyl-3-methylene-; 2-Butanethiol, 4-methoxy-2-methyl-; Pentanoic acid, 2-methyl-, ethyl ester; Bicyclo[3.1.0]hexane, 4-methylene-1-(1-methylethyl)-; Bicyclo[3.1.1]heptane, 6,6-dimethyl-2-methylene-; 1-Butanol, 3-methyl-, 1-propanoate; 1,6-Octadiene, 7-methyl-3-methylene-; Octanal; 2H-Pyran, 2-ethenyltetrahydro-2,6,6-trimethyl-; 2-Octanone; Hexanoic acid, ethyl ester; 2-Oxabicyclo[2.2.2]octane, 1,3,3-trimethyl-; Benzene, 1-methyl-4-(1-methylethyl)-; Benzene, 1-methoxy-4-methyl-; 1,3,6-Octatriene, 3,7-dimethyl-; Cyclohexene, 1-methyl-4-(1-methylethenyl)-; Cyclohexene, 1-methyl-4-(1-methylethenyl)-, (4R)—; 3-Octanone; Undecanal, 2-methyl-; Acetic acid, hexyl ester; 5-Hepten-2-one, 6-methyl-; 2-Hepten-4-one, 5-methyl-; 3-Hexen-1-ol, 1-acetate, (3Z)—; 3-Hexen-1-ol, 1-acetate; Propanoic acid, 2-hydroxy-, ethyl ester; Butanoic acid, 2-methylbutyl ester; Butanoic acid, 3-methylbutyl ester; 1,4-Cyclohexadiene, 1-methyl-4-(1-methylethyl)-; Thiazole, 2-(2-methylpropyl)-; 3-Hexen-1-ol, (3Z)—; Benzaldehyde; Butanoic acid, 3-oxo-, ethyl ester; 2-Hexen-1-ol, (2E)-; 2-Hexen-1-ol, (2Z)—; Cyclohexane, 3-ethoxy-1,1,5-trimethyl-, cis-(9CI); 2-Pentanone, 4-mercapto-4-methyl-; 2,4,6-Octatriene, 2,6-dimethyl-, (4E,6E)-; Oxirane, 2,2-dimethyl-3-(3-methyl-2,4-pentadien-1-yl)-; 4,7-Octadienoic acid, methyl ester, (4E)-; Carbonic acid, (3Z)-3-hexen-1-yl methyl ester; Hexanoic acid, 2-propen-1-yl ester; 5-Heptenal, 2,6-dimethyl-; Heptanoic acid, ethyl ester; 3-Cyclohexene-1-carboxaldehyde, 2,4-dimethyl-; Benzene, (2,2-dimethoxyethyl)-; 2H-Pyran, tetrahydro-4-methyl-2-(2-methyl-1-propen-1-yl)-; 3-Nonanone; Benzonitrile; 3-Octanol; 1-Hexanol, 3,5,5-trimethyl-, 1-acetate; 4-Heptanol, 2,6-dimethyl-, 4-acetate; Hexanoic acid, 2-methylpropyl ester; Propanoic acid, 2-methyl-, hexyl ester; Cyclohexanecarboxylic acid, 1,4-dimethyl-, methyl ester, trans-; Benzeneacetaldehyde; Butanoic acid, 3-hydroxy-, ethyl ester; Propanedioic acid, 1,3-diethyl ester; Benzoic acid, methyl ester; 1,3,5-Undecatriene; 4-Decenal, (4E)-; 1,3-Dioxane, 2-butyl-4,4,6-trimethyl-; 2-Heptanol, 2,6-dimethyl-; Ethanone, 1-phenyl-; Benzeneacetaldehyde, α-methyl-; Propanoic acid, 2-methyl-, 1,3-dimethyl-3-buten-1-yl ester; 2,6-Nonadienal, (2E,6Z)—; Pyrazine, 2-methoxy-3-(2-methylpropyl)-; Formic acid, phenylmethyl ester; Benzene, 1-methoxy-4-propyl-; Cyclohexanone, 5-methyl-2-(1-methylethyl)-, (2R,5R)-rel-; Cyclohexanone, 5-methyl-2-(1-methylethyl)-, (2R,5S)-rel-; 2-Nonenal; Cyclohexanone, 2-ethyl-4,4-dimethyl-; Benzene, 1,4-dimethoxy-; Benzene, 1-(ethoxymethyl)-2-methoxy-; Bicyclo[2.2.1]heptan-2-one, 1,7,7-trimethyl-; 2-Hexene, 6,6-dimethoxy-2,5,5-trimethyl-; Decanal; Benzenepropanal, β-methyl-; Benzenemethanol, α-methyl-, 1-acetate; Acetic acid, nonyl ester; Ethanone, 1-(4-methylphenyl)-; 2H-Pyran, 6-butyl-3,6-dihydro-2,4-dimethyl-; Propanoic acid, 2-methyl-, (3Z)-3-hexen-1-yl ester; Benzoic acid, ethyl ester; 3-Octanol, 3,7-dimethyl-, 3-acetate; 1-Hexanol, 5-methyl-2-(1-methylethyl)-, 1-acetate; Cyclohexanol, 3,3,5-trimethyl-, (1R,5R)-rel-; 2-Hexenal, 5-methyl-2-(1-methylethyl)-; 7-Octen-2-ol, 2,6-dimethyl-; Acetic acid, phenylmethyl ester; Cyclohexanone, 2-(1-methylpropyl)-; 3-Octen-1-ol, (3Z)—; Heptanoic acid, 2-propen-1-yl ester; Benzenemethanol; Butanoic acid, 2-methyl-, hexyl ester; 2(3H)-Furanone, 5-ethyldihydro-; Cyclohexaneethanol, 1-acetate; 2-Nonenoic acid, methyl ester; Butanoic acid, (3Z)-3-hexen-1-yl ester; 2-Octynoic acid, methyl ester; 1,3-Oxathiane, 2-methyl-4-propyl-, (2R,4S)-rel-; Heptanal, 6-methoxy-2,6-dimethyl-; Bicyclo[2.2.1]heptan-2-ol, 1,3,3-trimethyl-, 2-acetate; 1,6-Octadien-3-ol, 3,7-dimethyl-, 3-acetate; 2-Octanol, 2,6-dimethyl-; 1-Octanol; 3-Cyclohexene-1-methanethiol, α,α,4-trimethyl-; Cyclohexanemethanol, α,α,4-trimethyl-, 1-acetate; Cyclohexanol, 2-(1,1-dimethylethyl)-, 1-acetate; Cyclohexanol, 4-(1,1-dimethylethyl)-, 1-acetate; Pyrazine, 2-methoxy-3-(1-methylpropyl)-; Cyclohexanol, 5-methyl-2-(1-methylethenyl)-, (1R,2S,5R)—; 2-Undecanone; Benzenepropanol, α,α-dimethyl-; Bicyclo[2.2.1]heptan-2-ol, 1,7,7-trimethyl-, 2-acetate, (1R,2R,4R)-rel-; 1,6-Octadien-3-ol, 3,7-dimethyl-; Benzeneacetic acid, ethyl ester; Benzeneethanol, α,α-dimethyl-; Cyclopropanecarboxylic acid, (3Z)-3-hexen-1-yl ester; 3-Cyclohexene-1-methanol, 3,5-dimethyl-, 1-acetate; Undecanal; Ethanone, 1-(3-cycloocten-1-yl)-; Cyclohexanone, 4-(1,1-dimethylethyl)-; 6-Nonen-1-ol, (6Z)—; Benzene, (2-butoxyethyl)-; Bicyclo[3.1.1]hept-3-en-2-one, 4,6,6-trimethyl-; Cyclohexanecarboxylic acid, 2,2,6-trimethyl-, ethyl ester, (1R,6S)-rel-; Benzeneethanol; 2,6-Octadienal, 3,7-dimethyl-, (2Z)—; 2,6-Octadienal, 3,7-dimethyl-; Cyclohexanol, 5-methyl-2-(1-methylethyl)-, 1-acetate, (1R,2S,5R)-rel-; Benzoic acid, 2-hydroxy-, methyl ester; Benzene, 1-methoxy-4-(1E)-1-propen-1-yl-; 2,6-Octadiene, 1,1-dimethoxy-3,7-dimethyl-; Cyclohexanemethanol, a,3,3-trimethyl-, 1-formate; 2-Decenal, (2E)-; 3-Cyclopentene-1-acetonitrile, 2,2,3-trimethyl-; 2-Cyclohexen-1-one, 2-methyl-5-(1-methylethenyl)-, (5R)—; Cyclohexanone, 4-(1,1-dimethylpropyl)-; 6,10-Dioxaspiro[4.5]decane, 8,8-dimethyl-7-(1-methylethyl)-; 2-Cyclohexen-1-one, 3-methyl-5-propyl-; Benzonitrile, 4-(1-methylethyl)-; 2,6-Nonadienenitrile; Butanoic acid, 2-methyl-, (3Z)-3-hexen-1-yl ester; Benzene, 1-(cyclopropylmethyl)-4-methoxy-; 2-Nonynoic acid, methyl ester; Acetic acid, 2-phenylethyl ester; Cyclohexanol, 2-(1,1-dimethylethyl)-; 2,6-Nonadien-1-ol; Propanoic acid, 2-methyl-, phenylmethyl ester; Bicyclo[2.2.1]heptan-2-ol, 1,2,3,3-tetramethyl-, (1R,2R,4S)-rel-; Benzaldehyde, 4-(1-methylethyl)-; 2,5-Octadien-4-one, 5,6,7-trimethyl-, (2E)-; 3-Cyclohexen-1-ol, 4-methyl-1-(1-methylethyl)-; 3-Cyclohexene-1-methanol, 2,4,6-trimethyl-; Pentanoic acid, (3Z)-3-hexen-1-yl ester; Bicyclo[2.2.1]heptan-2-ol, 1,7,7-trimethyl-, 2-propanoate, (1R,2R,4R)-rel-; Benzene, 1-methyl-4-(1-methylethyl)-2-(1-propen-1-yl)-; 3-Cyclohexene-1-propanal, β,4-dimethyl-; 1-Hexanol, 5-methyl-2-(1-methylethyl)-, (2R)—; 3-Heptanone, 5-methyl-, oxime; 2(3H)-Furanone, 5-butyldihydro-; 1-Nonanol; Acetic acid, 2-(3-methylbutoxy)-, 2-propen-1-yl ester; Bicyclo[2.2.1]heptan-2-ol, 1,7,7-trimethyl-, (1S,2R,4S)—; Bicyclo[2.2.1]heptan-2-ol, 1,7,7-trimethyl-, (1R,2R,4R)-rel-; Cyclohexanol, 2-(1,1-dimethylpropyl)-, 1-acetate; 3-Cyclohexene-1-methanol, α,α,4-trimethyl-, 1-acetate; Cyclohexanemethanol, α,α,4-trimethyl-; 10-Undecenal; 2,4-Cyclohexadiene-1-carboxylic acid, 2,6,6-trimethyl-, ethyl ester; 1-Octanol, 3,7-dimethyl-; Furan, tetrahydro-2,4-dimethyl-4-phenyl-; Benzene, [2-(3-methylbutoxyl)ethyl]-; Butanoic acid, phenylmethyl ester; Benzoic acid, 2-hydroxy-, ethyl ester; Cyclohexanol, 4-(1,1-dimethylethyl)-; 1,6-Octadien-3-ol, 3,7-dimethyl-, 3-formate; Dodec anal; 3,6-Nonadien-1-ol, (3Z,6Z)—; 3,6-Nonadien-1-ol; Decanenitrile; Cyclohexanol, 5-methyl-2-(1-methylethyl)-, (1R,2S,5R)—; Propanoic acid, 2-methyl-, 4-methylphenyl ester; Propanoic acid, 2-methyl-, (1R,2S,4R)-1,7,7-trimethylbicyclo[2.2.1]hept-2-yl ester, rel-; Acetaldehyde, 2-(4-methylphenoxy)-; 2-Butenoic acid, 2-methyl-, (3Z)-3-hexen-1-yl ester, (2E)-; Bicyclo[3.1.1]hept-2-ene-2-propanal, 6,6-dimethyl-; 2-Nonanol, 6,8-dimethyl-; Cyclohexanol, 1-methyl-3-(2-methylpropyl)-; 1H-Indole; 2-Undecenal; 2H-Pyran-2-one, 4,6-dimethyl-; 3-Cyclohexene-1-methanol, α,α,4-trimethyl-; 3-Hepten-2-one, 3,4,5,6,6-pentamethyl-, (3Z)—; 2(3H)-Furanone, 5-butyldihydro-4-methyl-; 7-Octen-2-ol, 2,6-dimethyl-, 2-acetate; 2-Propenal, 3-phenyl-; 1,6-Octadien-3-ol, 3,7-dimethyl-, 3-propanoate; 1,6-Nonadien-3-ol, 3,7-dimethyl-, 3-acetate; Cyclopentanone, 2,2,5-trimethyl-5-pentyl-; 2,6-Octadien-1-ol, 3,7-dimethyl-, 1-acetate, (2Z)—; 2,6-Octadien-1-ol, 3,7-dimethyl-, 1-acetate, (2E)-; Undecane, 1,1-dimethoxy-2-methyl-; Benzenemethanol, α-methylene-, 1-acetate; Benzaldehyde, 4-methoxy-; Cyclohexanol, 5-methyl-2-(1-methylethenyl)-, 1-acetate, (1R,2S,5R)—; 6-Octenenitrile, 3,7-dimethyl-; 6-Octen-2-ol, 2,6-dimethyl-; Benzene, 1,1′-oxybis-; Benzoic acid, butyl ester; 5,8-Methano-2H-1-benzopyran, 6-ethylideneoctahydro-; Cyclohexanepropanol, α,α-dimethyl-; Benzenepropanal, β-methyl-3-(1-methylethyl)-; Benzenemethanol, 4-methoxy-, 1-acetate; Phenol, 2-ethoxy-4-methyl-; Benzene, [2-(1-propoxyethoxy)ethyl]-; 7-Octen-1-ol, 3,7-dimethyl-; Bicyclo[4.3.1]decane, 3-methoxy-7,7-dimethyl-10-methylene-; Propanoic acid, 2-(1,1-dimethylpropoxy)-, propyl ester, (2S)—; Benzoic acid, 2-(methylamino)-, methyl ester; 6-Octen-1-ol, 3,7-dimethyl-, (3S)—; 7-Octen-2-ol, 2-methyl-6-methylene-; 4,6-Octadien-3-ol, 3,7-dimethyl-; 5-Oxatricyclo[8.2.0.04,6]dodecane, 4,9,12,12-tetramethyl-; 2-Cyclohexene-1-carboxylic acid, 2-ethyl-6,6-dimethyl-, ethyl ester; 3-Buten-2-one, 4-(2,6,6-trimethyl-1-cyclohexen-1-yl)-, (3E)-; 4,7-Methano-1H-inden-5-ol, octahydro-, 5-acetate; Benzoic acid, 2-amino-, methyl ester; Spiro[1,3-dioxolane-2,8′(5′H)-[2H-2,4a]methanonaphthalene], hexahydro-1′,1′,5′,5′-tetramethyl-, (2'S,4′aS,8′aS)-(9CI); Spiro[1,3-dioxolane-2,8′(5′H)-[2H-2,4a]methanonaphthalene], hexahydro-1′,1′,5′,5′-tetramethyl-; 3-Buten-2-one, 4-(2,6,6-trimethyl-2-cyclohexen-1-yl)-, (3E)-; Benzeneethanol, α,α-dimethyl-, 1-acetate; 4,7-Methano-1H-inden-5-ol, 3α,4,5,6,7,7a-hexahydro-, 5-acetate; 6-Octen-1-ol, 3,7-dimethyl-, 1-acetate; 2H-Pyran, tetrahydro-2-methyl-4-methylene-6-phenyl-; Bicyclo[3.3.1]nonane, 2-ethoxy-2,6,6-trimethyl-9-methylene-; 2,6-Octadien-1-ol, 3,7-dimethyl-, (2E)-; Bicyclo[7.2.0]undec-4-ene, 4,11,11-trimethyl-8-methylene-, (1R,4E,9S)—; 1H-3α,7-Methanoazulene, octahydro-6-methoxy-3,6,8,8-tetramethyl-, (3R,3aS,6S,7R,8aS)-; Bicyclo[7.2.0]undec-4-ene, 4,11,11-trimethyl-8-methylene-, (1R,4E,9S)—; 1H-Inden-1-one, 2,3-dihydro-2,3,3-trimethyl-; 2-Propanol, 1,1′-oxybis-; 2-Octanol, 7-methoxy-3,7-dimethyl-; 4,9-Decadienal, 4,8-dimethyl-; Benzoic Acid; 3-Hexenoic acid, (3Z)-3-hexen-1-yl ester, (3Z)—; Bicyclo[2.2.1]hept-5-ene-2-carboxylic acid, 3-(1-methylethyl)-, ethyl ester, (1R,2S,3S,4S)-rel-; 2-Propen-1-ol, 3-phenyl-; Propanoic acid, 2-methyl-, 1-ethenyl-1,5-dimethyl-4-hexen-1-yl ester; Ethanol, 2-phenoxy-, 1-propanoate; 2-Propenoic acid, 3-phenyl-, methyl ester; Benzenepropanal, 2-ethyl-α,α-dimethyl-; Propanoic acid, decyl ester; Benzene, 1,2-dimethoxy-4-(1-propen-1-yl)-; 3-Decen-5-ol, 4-methyl-; Phenol, 2-methoxy-4-(2-propen-1-yl)-; 1-Propanone, 1-[2-methyl-5-(1-methylethyl)-2-cyclohexen-1-yl]-; 1,3-Benzodioxole-5-carboxaldehyde; 2-Dodecenal; 2-Dodecenal, (2E)-; Benzenepropanal, 4-methoxy-α-methyl-; 1,4-Cyclohexanedicarboxylic acid, 1,4-dimethyl ester; 2-Buten-1-one, 1-(2,6,6-trimethyl-3-cyclohexen-1-yl)-; 2-Butanone, 4-(2,6,6-trimethyl-1-cyclohexen-1-yl)-; 2-Propenenitrile, 3-phenyl-, (2E)-; Propanoic acid, 2-methyl-, 2-phenylethyl ester; 2-Cyclopenten-1-one, 3-methyl-2-(2Z)-2-penten-1-yl-; Acetaldehyde, 2-[(3,7-dimethyl-6-octen-1-yl)oxy]-; 1-Cyclohexene-1-ethanol, 4-(1-methylethyl)-, 1-formate; 2,4-Decadienoic acid, ethyl ester, (2E,4Z)—; 2-Propen-1-ol, 3-phenyl-, 1-acetate; Naphtho[2,1-b]furan, dodecahydro-3a,6,6,9a-tetramethyl-, (3aR,5aS, 9aS,9bR)-; Benzenepropanal, 4-(1,1-dimethylethyl)-; Naphtho[2,1-b]furan, dodecahydro-3a,6,6,9a-tetramethyl-; 1,4-Methanonaphthalen-5(1H)-one, 4,4a,6,7,8,8a-hexahydro-; Dodecanoic acid, 12-hydroxy-, λ-lactone (6CI,7CI); 1,12-; Cyclohexanepropanoic acid, 2-propen-1-yl ester; 2(3H)-Furanone, 5-hexyldihydro-5-methyl-; 2,6-Nonadienenitrile, 3,7-dimethyl-; 10-Undecenoic acid, ethyl ester; Benzenepropanal, α-methyl-4-(1-methylethyl)-; 1-Oxaspiro[4.5]decan-2-one, 8-methyl-; 2(3H)-Furanone, dihydro-5-pentyl-; 2(3H)-Furanone, 5-hexyldihydro-; 2-Buten-1-one, 1-(2,6,6-trimethyl-2-cyclohexen-1-yl)-, (2E)-; 2-Buten-1-one, 1-(2,4,4-trimethyl-2-cyclohexen-1-yl)-, (2E)-; 2H-Pyran-2-one, tetrahydro-6-pentyl-; Benzenepropanal, 4-ethyl-α,α-dimethyl-; 1,3-Benzodioxole, 5-(diethoxymethyl)-; 4-Penten-1-one, 1-(5,5-dimethyl-1-cyclohexen-1-yl)-; Bicyclo[3.1.1]hept-2-ene-2-ethanol, 6,6-dimethyl-, 2-acetate; 2-Propenoic acid, 3-phenyl-, ethyl ester; 1,3-Dioxane, 2,4,6-trimethyl-4-phenyl-; Cyclododecane, (methoxymethoxy)-; Bicyclo[3.1.1]hept-2-ene-2-propanal, α,α,6,6-tetramethyl-; Benzeneacetonitrile, 4-(1,1-dimethylethyl)-; 2-Buten-1-one, 1-(2,6,6-trimethyl-1-cyclohexen-1-yl)-; 1,4-Methanonaphthalen-6(2H)-one, octahydro-7-methyl-; Bicyclo[3.2.1]octan-8-one, 1,5-dimethyl-, oxime; Benzenepentanol, 7-methyl-; Cyclohexene, 4-(1,5-dimethyl-4-hexen-1-ylidene)-1-methyl-; Phenol, 2-methoxy-4-propyl-; Benzoic acid, 2-hydroxy-, 2-methylpropyl ester; 2H-1-Benzopyran-2-one, octahydro-; Cyclohexanone, 2-(1-mercapto-1-methylethyl)-5-methyl-; 2-Oxiranecarboxylic acid, 3-methyl-3-phenyl-, ethyl ester; 3-Cyclohexene-1-carboxaldehyde, 4-(4-methyl-3-penten-1-yl)-; Propanoic acid, 2-methyl-, 2-phenoxyethyl ester; Indeno[1,2-d]-1,3-dioxin, 4,4a,5,9b-tetrahydro-; 2H-Pyran-4-ol, tetrahydro-4-methyl-2-(2-methylpropyl)-; Cyclohexanebutanal, α,2,6,6-tetramethyl-; 1,6-Nonadien-3-ol, 3,7-dimethyl-; 3-Buten-2-one, 4-(2,2,6-trimethyl-7-oxabicyclo[4.1.0]hept-1-yl)-; Phenol, 2-methoxy-4-(1-propen-1-yl)-; 2(3H)-Furanone, 5-hexyldihydro-4-methyl-; 1-Penten-3-one, 1-(2,6,6-trimethyl-2-cyclohexen-1-yl)-; 2-Buten-1-one, 1-(2,6,6-trimethyl-1,3-cyclohexadien-1-yl)-; 2-Cyclopenten-1-one, 2-hydroxy-3-methyl-; Propanoic acid, 2,2-dimethyl-, 2-phenylethyl ester; Dodecanenitrile; 6-Octen-1-ol, 3,7-dimethyl-, 1-propanoate; Benzenepentanal, β-methyl-; Acetic acid, 2-phenoxy-, 2-propen-1-yl ester; Benzenepropanal, 4-(1,1-dimethylethyl)-α-methyl-; 4,7-Methano-1H-indene-2-carboxaldehyde, octahydro-5-methoxy-; Pentitol, 1,5-anhydro-2,4-dideoxy-2-pentyl-, 3-acetate; Cyclododecane, (ethoxymethoxy)-; 3-Buten-2-one, 4-(2,5,6,6-tetramethyl-2-cyclohexen-1-yl)-; Quinoline, 6-(1-methylpropyl)-; Carbonic acid, 4-cycloocten-1-yl methyl ester; 1H-Indene-5-propanal, 2,3-dihydro-3,3-dimethyl-; Ethanone, 1-(3-methyl-2-benzofuranyl)-; 3-Cyclohexene-1-carboxaldehyde, 1-methyl-3-(4-methyl-3-penten-1-yl)-; 6-Oxabicyclo[3.2.1]octane, 5-methyl-1-(2,2,3-trimethyl-3-cyclopenten-1-yl)-; 2H-Pyran-2-one, tetrahydro-6-(3-penten-1-yl)-; 2,4,7-Decatrienoic acid, ethyl ester; Butanoic acid, 3-methyl-, 2-phenylethyl ester; Spiro[1,4-methanonaphthalene-2(1H),2′-oxirane], 3,4,4a,5,8,8a-hexahydro-3′,7-dimethyl-; Ethanol, 2-[[(1R,2R,4R)-1,7,7-trimethylbicyclo[2.2.1]hept-2-yl]oxy]-, rel-; Phenol, 2-methoxy-4-(1-propen-1-yl)-, 1-acetate; 2H-Indeno[4,5-b]furan, decahydro-2,2,6,6,7,8,8-heptamethyl-; Acetic acid, 2-(cyclohexyloxy)-, 2-propen-1-yl ester; Octanal, 7-hydroxy-3,7-dimethyl-; Naphtho[2,1-b]furan, 9b-ethyldodecahydro-3a,7,7-trimethyl-; 1,6-Heptadien-3-one, 2-cyclohexyl-; 5-Thiazoleethanol, 4-methyl-; 1-Penten-3-one, 1-(2,6,6-trimethyl-2-cyclohexen-1-yl)-; 3-Buten-2-one, 3-methyl-4-(2,6,6-trimethyl-2-cyclohexen-1-yl)-; 1,4-Cyclohexanedicarboxylic acid, 1,4-diethyl ester; 2(3H)-Furanone, 5-heptyldihydro-; 1,3-Benzodioxole-5-propanal, α-methyl-; 4H-Inden-4-one, 1,2,3,5,6,7-hexahydro-1,1,2,3,3-pentamethyl-; Cyclohexanone, 4-(1-ethoxyethenyl)-3,3,5,5-tetramethyl-; Benzenepropanenitrile, α-ethenyl-α-methyl-; 9-Undecenal, 2,6,10-trimethyl-; Pyridine, 2-(3-phenylpropyl)-; Indeno[1,2-d]-1,3-dioxin, 4,4a,5,9b-tetrahydro-2,4-dimethyl-; Propanoic acid, 2-methyl-, 3a,4,5,6,7,7a-hexahydro-4,7-methano-1H-inden-5-yl ester; 1-Naphthalenol, 1,2,3,4,4a,7,8,8a-octahydro-2,4a,5,8a-tetramethyl-, 1-formate; Benzenepropanol, β,β,3-trimethyl-; 2-Cyclohexen-1-one, 4-(2-buten-1-ylidene)-3,5,5-trimethyl-; 3-Hexen-1-ol, 1-benzoate, (3Z)—; Benzaldehyde, 4-hydroxy-3-methoxy-; 1H-3a,7-Methanoazulen-6-ol, octahydro-3,6,8,8-tetramethyl-, 6-acetate, (3R,3aS,6R,7R,8aS)-; 4,7-Methano-1H-inden-6-ol, 3a,4,5,6,7,7a-hexahydro-8,8-dimethyl-, 6-propanoate; 2-Oxiranecarboxylic acid, 3-phenyl-, ethyl ester; 4H-4a,9-Methanoazuleno[5,6-d]-1,3-dioxole, octahydro-2,2,5,8,8,9a-hexamethyl-(4aR,5R,7aS,9R)—; Butanoic acid, 1,1-dimethyl-2-phenylethyl ester; Cyclododeca[c]furan, 1,3,3a,4,5,6,7,8,9,10,11,13a-dodecahydro-; Benzenebutanenitrile, α,α,γ-trimethyl-; 2-Butanone, 4-(1,3-benzodioxol-5-yl)-; Benzoic acid, 4-hydroxy-3-methoxy-, methyl ester; 3-Cyclopentene-1-butanol, β,2,2,3-tetramethyl-2-Methyl-4-(2,2,3-trimethyl-3-cyclopenten-1-yl)butanol; 2-Butenal, 2-methyl-4-(2,6,6-trimethyl-1-cyclohexen-1-yl)-; 2-Naphthalenol, decahydro-2,5,5-trimethyl-; 1,7-Octanediol, 3,7-dimethyl-; 2H-1-Benzopyran-2-one; 1,3-Dioxolane, 2-[6-methyl-8-(1-methylethyl)bicyclo[2.2.2]oct-5-en-2-yl]-; Propanoic acid, 2,2-dimethyl-, 3a,4,5,6,7,7a-hexahydro-4,7-methano-1H-inden-6-yl ester; Butanoic acid, (2E)-3,7-dimethyl-2,6-octadien-1-yl ester; 2-Butanone, 4-(4-hydroxyphenyl)-; 10-Undecenoic acid, butyl ester; and combinations thereof.

Additional examples of fragrance components include musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, limonenem, terpinene gamma, calone, helional, and mixtures thereof

The solvent component can range from about 30 wt % to about 95 wt % of the fragrance composition, about 40 wt % to about 90 wt % of the fragrance composition, less than, equal to, or greater than about 40 wt %, 45, 50, 55, 60, 65, 70, 75, 80, 85, 90, or 95 wt %. The solvent component can include a compound according to Formula I.

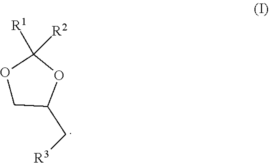

In Formula I, R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof. In some examples, R¹, R², and R³ are independently selected from the group consisting of —H, —OH, —CH₃, substituted or unsubstituted (C₂-C₁₀)alkyl, (C₂-C₁₀)alkeneyl, (C₃-C₁₀)cycloalkyl, (C₁-C₁₀)aryl, (C₁-C₁₀)alkoxyl, and a combination thereof. In some examples, R¹, R², and R³ are independently selected from the group consisting of —H, —OH, —CH₃, substituted or unsubstituted (C₂-C₆)alkyl, (C₂-C₆)alkeneyl, (C₃-C₆)cycloalkyl, (C₁-C₆)aryl, (C₁-C₆)alkoxyl, and a combination thereof. In some examples, R¹ and R² are each —CH₃ and R³ is —OH such that the compound is 2,2-dimethyl-1,3-dioxolan-4-ylmethanol, as shown in Formula II.

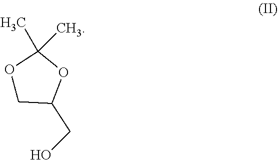

In further embodiments, the solvent component can include a mixture of the compound of Formula I and a second component. The second component can be an organic component. For example, the second component can be ethanol. When the solvent component is a mixture that includes the compound of Formula II, the compound of Formula II is in a range of from about 20 wt % to about 90 wt % of the of the fragrance composition, about 50 wt % to about 60 wt % of fragrance composition, less than, equal to, or greater than about 20 wt %, 25, 30, 35, 40, 45, 50, 55, 60, 65, 70, 75, 80, 85, or about 90 wt %. In specific embodiments, where the organic component is ethanol, the ethanol is in a range of from about 5 wt % to about 55 wt % of the fragrance composition, about 20 wt % to about 40 wt % of the fragrance composition, less than, equal to, or greater than about 5 wt %, 10, 15, 20, 25, 30, 35, 40, 45, 50, or about 55 wt %.

Using the compound of Formula II as a solvent can act to substantially replace ethanol as a solvent. However, the compound of Formula II can be subject to hydrolysis, which can lead to the production of acetone. If acetone is produced in too high of a level, the odor associated with the acetone can render the fragrance component. If amounts of ethanol as described herein above are present in the fragrance composition, the amount of water in the fragrance composition can come from the ethanol. Acceptable amounts of water in the fragrance composition can range from about 0.5 wt % to about 15 wt %, about 1 wt % to about 5 wt %, less than, equal to, or greater than about 0.5 wt %, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or about 15 wt %. The amount of water can be controlled such that acetone is present in an amount of less than about 5 wt % of the fragrance composition, less than 4 wt %, less than 3 wt %, less than 2 wt %, less than 1 wt %, or 0% of the fragrance composition.

The pH of the fragrance composition is kept at or below 7. It was found that increasing the pH to an alkaline value above 7, renders the composition unsuitable as a fragrance composition. More specifically a pH of the fragrance composition is kept in a range of from about 4 to about 7, about 4 to about 6.5, about 6 to about 6.5, less than, equal to, or greater than about 4, 4.5, 5, 5.5, 6, or 6.5. In some aspects, it was found that a pH from 6.5 to 7 resulted in less acetone being formed. The pH of the fragrance composition can be controlled or maintained using a buffer system comprises. Where present, the buffer system can be in a range of from about 0.01 wt % to about 2 wt % of the fragrance composition, about 0.01 wt % to about 0.05 wt % of the fragrance composition, less than, equal to, or greater than about 0.01, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, or about 2 wt %. Ty buffer system can include triethanolamine, citric acid, tetrahydroxypropyl ethylenediamine, or a mixture thereof. In specific aspects, the buffer system can be free of triethanolamine. In some specific aspects, the buffer system includes tetrahydroxypropyl ethylenediamine.

### Examples

Various embodiments of the present invention can be better understood by reference to the following Examples which are offered by way of illustration. The present invention is not limited to the Examples given herein.

Various formulations were tested to monitor acetone formation and pH over the course of 8 weeks. The various formulations are shown in Table 1 and results are shown at Table 2:

TABLE 1
Formulations

| | Formulation | | | | | | |
|---|---|---|---|---|---|---|---|
| Component (Wt %) | 1 | 2 | 3 | 4 | 5 | 6 | 7 |
| Ethanol | 45 | 45 | 45 | 45 | 45 | 45 | 45 |
| 2,2-dimethyl-1,3- | 33 | 43 | 33 | 43 | 33 | 45 | 45 |
| dioxolan-4-ylmethanol | | | | | | | |
| Water | 10 | 0 | 9.5 | 0 | 11.8 | 0 | 0 |
| Citric Acid and | 0 | 0 | 0 | 0.06 | 0 | 0 | 0.06 |
| triethanolamine buffer | | | | | | | |
| Fragrance Component | 10 | 10 | 10 | 10 | 9 | 9 | 9 |
| UV Filter | 0.5 | 0.5 | 0.5 | 0.5 | 0.5 | 0.5 | 0.5 |

TABLE 2
Results

| Formula | Week 45° C. | Acetone (PPM) | pH |
|---|---|---|---|
| 1 | 0 | 50 | 6.33 |
| | 2 | 370 | 5.83 |
| | 4 | 740 | 5.85 |
| | 6 | 1090 | 5.79 |
| | 8 | 1440 | 5.84 |
| 2 | 0 | 20 | 6.1 |
| | 2 | 310 | 5.76 |
| | 4 | 630 | 5.77 |
| | 6 | 910 | 5.65 |
| | 8 | 1200 | 5.72 |
| 3 | 0 | 220 | 6.43 |
| | 2 | 1330 | 6.27 |
| | 4 | 2860 | 6.12 |
| | 6 | 4050 | 6.02 |
| | 8 | 5620 | 5.89 |
| 4 | 0 | 20 | 6.65 |
| | 2 | 310 | 6.39 |
| | 4 | 590 | 6.5 |
| | 6 | 870 | 6.44 |
| | 8 | 1220 | 6.45 |
| 5 | 0 | 1570 | 5.62 |
| | 2 | 10340 | 5.26 |
| | 4 | 19480 | 5.29 |
| | 6 | 26670 | 5.06 |
| | 8 | 35250 | 5.14 |
| 6 | 0 | 0 | 6.01 |
| | 2 | 200 | 5.93 |
| | 4 | 460 | 5.92 |
| | 6 | 690 | 6.34 |
| | 8 | 1220 | 5.91 |
| 7 | 0 | 20 | 6.47 |
| | 2 | 340 | 6.37 |
| | 4 | 680 | 6.4 |
| | 6 | 950 | 6.08 |
| | 8 | 4720 | 6.46 |

The formulations were further tested to monitor acetone formation over the course of one year as well as their stability. Stability refers to the acetone concentration (ppm) measured at 8 weeks at 45° C. The results are shown at Table 3:

| | Acetone | Acetone | Stability at |
|---|---|---|---|
| | Concentration | Concentration | 45° C. at |
| Formula | (ppm) at 0 weeks | (ppm) at 52 weeks | 8 weeks. |
| 1 | 47 | 570 | 1440 |
| 2 | 17 | 530 | 1200 |
| 3 | 219 | 2340 | 5620 |
| 4 | 18 | 290 | 1220 |
| 5 | 1608 | 18020 | 35250 |
| 6 | 15 | 400 | 1220 |
| 7 | 22 | 330 | 4720 |

Stability was assessed according to the following protocol. A plurality of samples were tested at various temperatures that a sample would be exposed to in use outside of the lab for a desired amount of time. Following the desired period of time, the sample was evaluated for its color and odor. If there was discoloration and/or unpleasant odor formation, the sample was deemed to be unstable.

The acetone concentrations discussed herein were determined according to the following protocol.

### Equipment, Reagents, and Solutions

| Apparatus & | |
|---|---|
| Materials | Model/Type |
| Gas Chromatograph | Agilent 7890 equipped with FID |
| Column | Stabilwax-MS RESTEK (10673) 30 m × 250 μm × |
| | 0.25 μm or equivalent (e.g DB-Wax, Agilent) |
| Analytical Balance | e.g. Mettler, accurate to 3 decimal places |
| Glass vial | 20 mL |
| GC vial | 2 mL |
| Reagents | Purity and Source (or Equivalent) |
| Absolute Ethanol | Min. 99.5%, GC or analytical grade |
| [CAS 64-17-5] | |
| Standard substances | Purity and Source (or Equivalent) |
| Acetone | Min 99.5%, GC or analytical grade |
| [CAS 67-64-1] | |

### Procedure

a) Stock Standard Solution (˜10000 ppm)
In a vial exactly 0.10 g of Acetone was weigh. An additional 9.90 g of ethanol was added. Cap and mix solution.

### b) Working Standard Solutions

In a vial, the following amounts of stock std and Ethanol were weight, capped and mixed well.

| Calibration | Target Weight | Target Weight | Approx. concentration |
|---|---|---|---|
| solution | of Stock STD, g | of Ethanol, g | (ppm) |
| CAL STD 1 | 1.00 | 9.00 | 1000 |
| CAL STD 2 | 1.75 | 5.00 | 3500 |
| CAL STD 3 | 3.00 | 5.00 | 6000 |

### c) Sample Preparation

An aliquot of the EDT to be tested was transferred directly into a GC vial and cap. No dilution or sample preparation was required.

### d) Instrument Conditions

$$Total runtime = 28 min$$

| Parameter | Setpoint | |
|---|---|---|
| Carrier Gas | Helium | |
| Column Flow rate | 0.7 mL/min - constant flow | |
| Injection temperature | 250° | C. |
| Injection mode | Split | |
| Split ratio | 20:1 | |
| Injection volume | 1 | microliter |
| Oven Temperature initial | 40° | C. |
| Oven Temperature Rate 1 | 5° | C./min |
| Oven Temperature T1 | 70° | C. |
| Oven Temperature Rate 2 | 15° | C./min |
| Oven Temperature T2 | 250° C. hold for 10 min | |
| Detector Temperature | 250° | C. |

| | |
|---|---|
| Flame | 30 mL H₂/400 mL Air/30 |
| (suggested conditions) | mL Makeup gas (N2) |

A 3-point calibration curve (linear, forced through zero) was generated using all calibration injections across the sequence.

### Standard Concentration

$$Acetone Concentration in Stock Std , ppm = S 1 = \frac{weight of Acetone , g}{weight of Ethanol + Acetone , g} \times {10}^{6} \\ Acetone Concentration in CAL Std , ppm = \frac{S 1 \times weight of stock std , g}{weight of Ethanol + Stock S td , g}$$

### Calibration Curve

The calibration curve was plotted and, using a linear regression forcing through zero, determine the slope, a, where y=ax.

### Sample Concentration

$$Acetone concentration in sample , ppm = \frac{{A}_{S}}{a}$$

Where: A_{S}=Area of Acetone peak in sample chromatogram
a=slope of calibration slope

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

### Exemplary Aspects

The following exemplary aspects are provided, the numbering of which is not to be construed as designating levels of importance:

Aspect 1 provides a fragrance composition comprising:
a fragrance component; and
a solvent component comprising a compound according to Formula I:

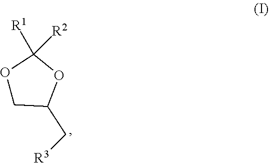

wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof.

Aspect 2 provides the fragrance composition of Aspect 1, wherein R¹ and R² are each —CH₃ and R³ is —OH such that the compound is 2,2-dimethyl-1,3-dioxolan-4-ylmethanol.

Aspect 3 provides the fragrance composition of any one of Aspects 1 or 2, wherein the composition comprises less than about 15 wt % of water.

Aspect 4 provides the fragrance composition of any one of Aspects 1-3, wherein the composition comprises about 0.5 wt % water to about 15 wt % water.

Aspect 5 provides the fragrance composition of any one of Aspects 1-4, wherein the composition comprises about 1 wt % water to about 5 wt % water.

Aspect 6 provides the fragrance composition of any one of Aspects 1-5, further comprising:
a buffer system; and
an optional colorant.

Aspect 7 provides the fragrance composition of any one of Aspects 1-6, wherein a pH of the fragrance composition is less than or equal to 7.

Aspect 8 provides the fragrance composition of any one of Aspects 1-7, wherein a pH of the fragrance composition is in a range of from about 4 to about 6.5.

Aspect 9 provides the fragrance composition of any one of Aspects 1-8, wherein a pH of the fragrance composition is in a range of from about 6 to about 6.5.

Aspect 10 provides the fragrance composition of any one of Aspects 1-9, wherein the fragrance component comprises musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, limonenem, terpinene gamma, calone, helional, and mixtures thereof.

Aspect 11 provides the fragrance composition of any one of Aspects 1-10, wherein the fragrance component comprises limonene, terpinene gamma, calone, helional, and mixtures thereof.

Aspect 12 provides the fragrance composition of any one of Aspects 1-11, wherein the fragrance component is in a range of from about 2 wt % to about 20 wt % of the fragrance composition.

Aspect 13 provides the fragrance composition of any one of Aspects 1-12, wherein the fragrance component is in a range of from about 5 wt % to about 15 wt % of the fragrance composition.

Aspect 14 provides the fragrance composition of any one of Aspects 6-13, wherein the buffer system comprises triethanolamine, citric acid, or a mixture thereof.

Aspect 15 provides the fragrance composition of any one of Aspects 6-14, wherein the buffer system is in a range of from about 0.01 wt % to about 2 wt % of the fragrance composition.

Aspect 16 provides the fragrance composition of any one of Aspects 6-15, wherein the buffer system is in a range of from about 0.01 wt % to about 0.05 wt % of the fragrance composition.

Aspect 17 provides the fragrance composition of any one of Aspects 1-16, wherein the solvent system is in a range of from about 30 wt % to about 95 wt % of the fragrance composition.

Aspect 18 provides the fragrance composition of any one of Aspects 1-17, wherein the solvent system is in a range of from about 40 wt % to about 90 wt % of the fragrance composition.

Aspect 19 provides the fragrance composition of any one of Aspects 1-18, wherein the solvent system comprises a mixture of 2,2-dimethyl-1,3-dioxolan-4-ylmethanol and an organic component.

Aspect 20 provides the fragrance composition of Aspect 19, wherein the organic component comprises ethanol.

Aspect 21 provides the fragrance composition of any one of Aspects 19 or 20, wherein the 2,2-dimethyl-1,3-dioxolan-4-ylmethanol is in a range of from about 20 wt % to about 90 wt % of the fragrance composition.

Aspect 22 provides the fragrance composition of any one of Aspects 19-21, wherein the 2,2-dimethyl-1,3-dioxolan-4-ylmethanol is in a range of from about 30 wt % to about 45 wt % of the fragrance composition.

Aspect 23 provides the fragrance composition of any one of Aspects 20-22, wherein the ethanol is less than 55 wt % of the fragrance composition.

Aspect 24 provides the fragrance composition of any one of Aspects 20-23, wherein the ethanol is in a range of from about 5 wt % to about 55 wt % of the fragrance composition.

Aspect 25 provides the fragrance composition of any one of Aspects 20-24, wherein the ethanol is in a range of from about 20 wt % to about 40 wt % of the fragrance composition.

Aspect 26 provides the fragrance composition of any one of Aspects 1-25, wherein the fragrance composition comprises less than 5 wt % acetone.

Aspect 27 provides the fragrance composition of any one of Aspects 1-26, wherein the fragrance composition is free of acetone.

Aspect 28 provides a fragrance composition comprising:
a fragrance component;
a solvent component comprising a compound according to Formula I:

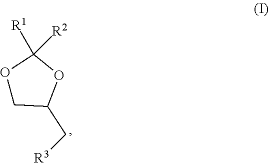

wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof,
a buffer system; and
an optional colorant.

Aspect 29 provides the fragrance composition of Aspect 28, wherein R¹ and R² are each —CH₃ and R³ is —OH such that the compound is 2,2-dimethyl-1,3-dioxolan-4-ylmethanol.

Aspect 30 provides the fragrance composition of any one of Aspects 28 or 29, wherein the composition comprises less than about 15 wt % of water.

Aspect 31 provides the fragrance composition of any one of Aspects 28 or 30, wherein the composition comprises about 0.5 wt % water to about 15 wt % water.

Aspect 32 provides the fragrance composition of any one of Aspects 28-31, wherein the composition comprises about 1 wt % water to about 5 wt % water.

Aspect 33 provides the fragrance composition of any one of Aspects 28-32, wherein a pH of the fragrance composition is less than or equal to 7.

Aspect 34 provides the fragrance composition of any one of Aspects 28-33, wherein a pH of the fragrance composition is in a range of from about 4 to about 6.5.

Aspect 35 provides the fragrance composition of any one of Aspects 28-34, wherein a pH of the fragrance composition is in a range of from about 6 to about 6.5.

Aspect 36 provides the fragrance composition of any one of Aspects 28-35, wherein the fragrance component comprises musk oil, civet, castoreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, coconut oil, salvia oil, clove oil, chamomile oil, costus oil, labdanum oil, broom extract, carrot seed extract, jasmine extract, mimosa extract, narcissus extract, olibanum extract, rose extract, acetophenonene and derivatives thereof, dimethylaniline derivatives, naphthaline derivatives, allyl caprate, alpha-amylcinnamic aldehyde, anethole, anisaldehyde, benzyl acetate, benzyl alcohol, benzyl propionate, borneol, cinnamyl acetate, cinnamyl alcohol, citral, citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl isovalerate, iso-eugenol, linalol, linalyl acetate, p-methylacetophenone, methyl anthranilate, methyl dihydrojasmonate, methyl eugenol, methyl-beta-naphthol ketone, methyl phenyl carbinyl acetate, musk ketol, musk xylol, 2,5,6-nonadienal, gamma-nonalactone, phenyl acetaldehyde dimethyl acetal, beta-phenylethyl alcohol, 3,3,5-trimethylcyclohexanol, gamma-undecalactone, undecenal, vanillin, metofluthrin, limonenem, terpinene gamma, calone, helional, and mixtures thereof.

Aspect 37 provides the fragrance composition of any one of Aspects 28-36, wherein the fragrance component comprises limonene, terpinene gamma, calone, helional, and mixtures thereof.

Aspect 38 provides the fragrance composition of any one of Aspects 28-37, wherein the fragrance component is in a range of from about 2 wt % to about 20 wt % of the fragrance composition.

Aspect 39 provides the fragrance composition of any one of Aspects 28-38, wherein the fragrance component is in a range of from about 5 wt % to about 15 wt % of the fragrance composition.

Aspect 40 provides the fragrance composition of any one of Aspects 28-39, wherein the buffer system comprises triethanolamine, citric acid, or a mixture thereof.

Aspect 41 provides the fragrance composition of any one of Aspects 28-40, wherein the buffer system is in a range of from about 0.01 wt % to about 2 wt % of the fragrance composition.

Aspect 42 provides the fragrance composition of any one of Aspects 28-41, wherein the buffer system is in a range of from about 0.01 wt % to about 0.05 wt % of the fragrance composition

Aspect 43 provides the fragrance composition of any one of Aspects 28-42, wherein the solvent system is in a range of from about 30 wt % to about 95 wt % of the fragrance composition.

Aspect 44 provides the fragrance composition of any one of Aspects 28-43, wherein the solvent system is in a range of from about 40 wt % to about 90 wt % of the fragrance composition.

Aspect 45 provides the fragrance composition of any one of Aspects 28-44, wherein the solvent system comprises a mixture of 2,2-dimethyl-1,3-dioxolan-4-ylmethanol and an organic component.

Aspect 46 provides the fragrance composition of Aspect 45, wherein the organic component comprises ethanol.

Aspect 47 provides the fragrance composition of any one of Aspects 45 or 46, wherein the 2,2-dimethyl-1,3-dioxolan-4-ylmethanol is in a range of from about 20 wt % to about 90 wt % of the fragrance composition.

Aspect 48 provides the fragrance composition of any one of Aspects 45-47, wherein the 2,2-dimethyl-1,3-dioxolan-4-ylmethanol is in a range of from about 30 wt % to about 45 wt % of the fragrance composition.

Aspect 49 provides the fragrance composition of any one of Aspects 46-48, wherein the ethanol is less than 55 wt % of the fragrance composition.

Aspect 50 provides the fragrance composition of any one of Aspects 46-49, wherein the ethanol is in a range of from about 5 wt % to about 55 wt % of the fragrance composition.

Aspect 51 provides the fragrance composition of any one of Aspects 46-50, wherein the ethanol is in a range of from about 20 wt % to about 40 wt % of the fragrance composition.

Aspect 52 provides the fragrance composition of any one of Aspects 28-51, wherein the fragrance composition comprises less than 5 wt % acetone.

Aspect 53 provides the fragrance composition of any one of Aspects 28-52, wherein the fragrance composition is free of acetone.

Aspect 54 provides a fragrance composition comprising:
a fragrance component in a range of from about 2 wt % to about 20 wt % of the fragrance composition;
a solvent component comprising a mixture of 2,2-dimethyl-1,3-dioxolan-4-ylmethanol and ethanol;
a buffer system; and
a colorant,
wherein the fragrance composition comprises less than about 15 wt % of water and a pH of the fragrance composition is below 7.

Aspect 55 provides an assembly comprising:
a container; and
the fragrance composition of any one of Aspects 1-54, disposed at least partially within the container.

Aspect 56 provides the fragrance composition of any one of Aspects 28-39, wherein the buffer system is free of triethanolamine.

Aspect 57 provides the fragrance composition of any one of Aspects 28-39 and 56, wherein the buffer system comprises tetrahydroxypropyl ethylenediamine.

1. A fragrance composition comprising:
a fragrance component; and
a solvent component comprising a compound according to Formula I: 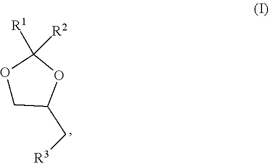
wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof.

2. The fragrance composition of claim 1, wherein R¹ and R² are each —CH₃ and R³ is —OH such that the compound is 2,2-dimethyl-1,3-dioxolan-4-ylmethanol.

3. The fragrance composition of claim 1, wherein the composition comprises less than about 15 wt % of water.

4. The fragrance composition of claim 1, further comprising:
a buffer system; and
an optional colorant.

5. The fragrance composition of claim 1, wherein a pH of the fragrance composition is in a range of from about 4 to about 6.5.

6-7. (canceled)

8. The fragrance composition of claim 1, wherein the fragrance component comprises limonene, terpinene gamma, calone, helional, and mixtures thereof.

9. The fragrance composition of claim 1, wherein the fragrance component is in a range of from about 2 wt % to about 20 wt % of the fragrance composition.

10. The fragrance composition of claim 1, wherein the buffer system comprises triethanolamine, citric acid, tetrahydroxypropyl ethylenediamine, or a mixture thereof.

11. The fragrance composition of claim 1, wherein the buffer system is free of triethanolamine.

12. The fragrance composition of claim 1, wherein the buffer system comprises tetrahydroxypropyl ethylenediamine.

13. The fragrance composition of claim 1, wherein the buffer system is in a range of from about 0.01 wt % to about 2 wt % of the fragrance composition.

14. The fragrance composition of claim 1, wherein the buffer system is in a range of from about 0.01 wt % to about 0.05 wt % of the fragrance composition.

15. The fragrance composition of claim 1, wherein the solvent system comprises a mixture of 2,2-dimethyl-1,3-dioxolan-4-ylmethanol and an organic component.

16. A fragrance composition comprising:
a fragrance component;
a solvent component comprising a compound according to Formula I: 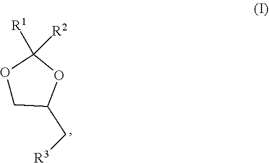
wherein R¹, R², and R³ are independently selected from the group consisting of —H, —OH, —CH₃, substituted or unsubstituted (C₂-C₂₀)alkyl, (C₂-C₂₀)alkeneyl, (C₃-C₂₀)cycloalkyl, (C₁-C₂₀)aryl, (C₁-C₂₀)alkoxyl, and a combination thereof;
a buffer system; and
an optional colorant.

17-31. (canceled)

32. The fragrance composition of claim 16, wherein the solvent system is in a range of from about 30 wt % to about 95 wt % of the fragrance composition.

33. The fragrance composition of claim 16, wherein the solvent system is in a range of from about 40 wt % to about 90 wt % of the fragrance composition.

34. The fragrance composition of claim 16, wherein the solvent system comprises a mixture of 2,2-dimethyl-1,3-dioxolan-4-ylmethanol and an organic component.

35. The fragrance composition of claim 34, wherein the organic component comprises ethanol.

36. The fragrance composition of claim 34, wherein the ethanol is less than 55 wt % of the fragrance composition.

37. A fragrance composition comprising:
a fragrance component in a range of from about 2 wt % to about 20 wt % of the fragrance composition;
a solvent component comprising a mixture of 2,2-dimethyl-1,3-dioxolan-4-ylmethanol and ethanol;
a buffer system; and
a colorant,
wherein the fragrance composition comprises less than about 15 wt % of water and a pH of the fragrance composition is below 7.

38. (canceled)
